(19) United States
(12) Patent Application Publication
(10) Pub. No.: US 2015/0080250 A1
(43) Pub. Date: Mar. 19, 2015

(54) SOLID SUPPORT AND METHOD FOR DETECTING AN ANALYTE IN A SAMPLE

(71) Applicant: ZBX Corporation, Toronto, Ontario (CA)

(72) Inventors: Qinwei Shi, Ontario (CA); Jing Xu, Ontario (CA)

(73) Assignee: ZBX Corporation, Toronto (CA)

(21) Appl. No.: 14/395,547

(22) PCT Filed: Apr. 19, 2013
(86) PCT No.: PCT/CA2013/000388

§ 371 Date: Oct. 20, 2014

Related U.S. Application Data
(60) Provisional application No. 61/636,066, filed on Apr. 20, 2012.

(51) Int. Cl. G01N 33/68; G01N 33/548; G01N 33/543

(52) U.S. Cl. CPC G01N 33/686; G01N 33/54393; G01N 33/548
USPC 506/9; 435/7.5; 436/501; 506/18

(57) ABSTRACT

A solid support for detecting the presence of an analyte in a sample at or above a predetermined threshold comprises: a competitive analyte within said solid support, wherein said competitive analyte is coupled to a first member of a binding pair; a labelled conjugate within said solid support, downstream of said competitive analyte, wherein said analyte and said competitive analyte compete for binding to said conjugate; and a capture reagent immobilized within said solid support, downstream of said conjugate, wherein said capture reagent comprises a second member of the binding pair; wherein the affinity of the first and second members of the binding pair for one another and the distance between the conjugate and the capture reagent and/or the conjugate and the competitive analyte within the solid support are selected so as to increase the threshold of the solid support for the analyte.

### FIELD OF THE INVENTION

The present invention relates to analyte detection. More specifically, the present invention relates to solid supports and methods for detecting an analyte in a sample at or above a predetermined threshold.

### BACKGROUND OF THE INVENTION

Generally, solid supports and methods for detecting analytes have the aim of increasing sensitivity because most analytes are present in samples at very low levels and the clinically relevant threshold for detection is typically in the ng/ml to low μg/ml range. However, certain analytes are present in samples at much higher amounts and the clinically relevant threshold is too high to be detected directly using conventional methods. Instead, typical methods of detecting such high concentration analytes involve dilution of the sample by the end user. Additionally, many conventional methods are not suitable to be directly used by the end user and the sample must instead be sent to a laboratory for determining the end result. This delay is problematic when analyte detection must occur rapidly.

For example, newborn calves can only acquire passive immunity to disease through the uptake of immunoglobulin (Ig) from colostrum. This transfer of colostral Ig is only possible within about the first day of the life of the calf. Termination of the calves' intestinal permeability occurs spontaneously at a progressively increasing rate after about 12 hours postpartum. The amount of Ig transfer varies widely, and it is recognized that up to about 40% of calves do not acquire adequate passive immunity. The resultant low Ig concentration in those calves is associated with a high rate of morbidity and mortality. However, the amount of Ig present in calves, regardless of whether or not they are in need of immune transfer is rather high, in the mg/ml range, and therefore outside of the range of direct detection by conventional methods.

Accordingly, there is a need to mitigate and/or overcome at least one deficiency of the prior art.

### SUMMARY OF THE INVENTION

The present invention relates, in aspects, to a solid support and one-step method for detecting the presence of an analyte in a sample at or above a predetermined threshold. The solid support and method described herein are useful in detecting analytes in a sample wherein the threshold is in the mg/ml range. Such solid supports and methods are thus useful in identifying newborn calves or other mammals that are in need of immune system transfer shortly after birth.

In accordance with an aspect, there is provided a solid support for detecting the presence of an analyte in a sample at or above a predetermined threshold, the solid support comprising:
a competitive analyte within said solid support, wherein said competitive analyte is coupled to a first member of a binding pair;
a labelled conjugate within said solid support, downstream of said competitive analyte, wherein said analyte and said competitive analyte compete for binding to said conjugate; and
a capture reagent immobilized within said solid support, downstream of said conjugate, wherein said capture reagent comprises a second member of the binding pair;
wherein the affinity of the first and second members of the binding pair for one another and the distance between the conjugate and the capture reagent and/or the conjugate and the competitive analyte within the solid support are selected so as to increase the threshold of the solid support for the analyte.

In an aspect, the solid support of claim 1, wherein the analyte is an immunoglobulin, such as IgG.

In an aspect, the conjugate is an antibody specific for the analyte.

In an aspect, the first member of said binding pair is biotin and the second member of said binding pair is streptavidin or avidin.

In an aspect, the sample is a biological sample selected from the group consisting of whole blood, serum, plasma, urine, milk, and colostrum.

In an aspect, the predetermined threshold is at least about 1 mg/ml, 3 mg/ml, 5 mg/ml, 10 mg/ml, 15 mg/ml, 20 mg/ml, 25 mg/ml, 30 mg/ml, 40 mg/ml, or 50 mg/ml.

In an aspect, the solid support further comprises a blocking capture reagent upstream of said competitive analyte.

In accordance with another aspect, there is provided a solid support for detecting the presence of an analyte in a sample at or above a predetermined threshold, the solid support comprising:
a blocking capture reagent immobilized within said solid support, wherein said blocking capture reagent binds to the analyte in the sample and thereby reduces the concentration of the analyte that is mobile;
a labelled conjugate within said solid support, downstream of said blocking capture reagent; and
a capture reagent immobilized within said solid support, downstream of said conjugate, wherein said capture reagent is a competitive analyte and wherein said analyte and said competitive analyte compete for binding to said conjugate;
wherein the distance between the conjugate and the capture reagent within the solid support is selected so as to increase the threshold of the solid support for the analyte.

In accordance with another aspect, there is provided a one-step method for detecting an analyte in a sample, the method comprising applying the sample to the solid support described herein, wherein a positive signal indicates that the analyte is absent or present in an amount below the predetermined threshold.

In accordance with another aspect, there is provided a one-step method for determining whether a newborn mammal is in need of immune transfer, the method comprising applying a biological sample from the newborn mammal or its mother to a solid support, said solid support comprising:
mammalian IgG coupled to a first member of a binding pair;
labelled anti-mammalian IgG antibody downstream of said mammalian IgG; and
a second member of the binding pair downstream of said anti-mammalian IgG;
wherein the affinity of the first and second members of the binding pair for one another and the distance between the anti-mammalian IgG antibody and the second member of the binding pair and/or the anti-mammalian IgG and the mammalian IgG are selected such that a positive signal results when the mammal has an IgG level at or below a predetermined threshold in the mg/ml range, indicating that the mammal is in need of immune transfer.

In an aspect, the first member of said binding pair is biotin and the second member of said binding pair is streptavidin or avidin.

In an aspect, the sample is derived from the newborn mammal and is selected from the group consisting of whole blood, serum, plasma, and urine.

In an aspect, the sample is derived from the mother of the newborn mammal and is selected from the group consisting of milk and colostrum.

In an aspect, the mammal is a cow, horse, alpaca, or llama.

In an aspect, the predetermined threshold is about 10 mg/ml.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from said detailed description.

### DESCRIPTION OF THE FIGURES

The present invention will be further understood from the following description with reference to the Figures, in which:

FIG. 1 shows a schematic view of a first aspect of a solid support as described herein; and

FIG. 2 shows a schematic view of a second aspect of a solid support as described herein.

### DETAILED DESCRIPTION OF THE INVENTION

Conventional solid support assays have been optimized to increase their sensitivity in order to reduce the threshold for detecting the presence of an analyte in a sample. This is because clinically relevant threshold levels of most analytes are quite low, such as in the pg/ml range or lower. Therefore, such conventional solid support assays are not directly useful in a one-step method for identifying threshold levels of analytes that are present in high amounts in samples, wherein the clinically relevant threshold levels of such analytes are much higher, such as, for example, in the mg/ml range.

When conventional solid support assays are used to detect analytes that are present in high amounts in samples, typically dilution of the sample is required prior to applying the sample to the solid support. “Dilution” means that a blood or urine sample, for example, is diluted with a suitable diluent prior to being applied to the solid support assay and is not used as-is directly from the body. Such assays are thus not considered “one-step” assays, as dilution of the sample is required. Dilution of samples can be the source of several problems, including at least contamination of the sample and inaccurate test results due to improper dilution of the sample. Moreover, dilution of the sample requires calculation and measurement by the end user, which can also lead to inaccurate test results.

In particular, the high sensitivity of conventional solid support assays has been a problem in quickly identifying newborn calves that are in need of immune transfer. Such calves must be identified as soon as possible because there is a very limited window in which immune transfer can effectively occur. Additionally, newborn calves generally have bovine IgG present in their blood in the mg/ml range, with a threshold of about 10 mg/ml IgG dividing the calves between those that require immune transfer (those with IgG below 10 mg/ml) and those that do not (those with IgG at or above 10 mg/ml). It has been difficult in the past to find a test that can be performed “cowside” in one step without dilution of the sample to provide a quick and accurate test result to determine which calves are below the threshold of the test and thus require immune transfer.

Accordingly, the present invention is directed to a solid support and one-step method for detecting the presence of an analyte in a sample at or above a predetermined threshold. Typically, the threshold is in the mg/ml range. Because the solid support described herein has been modified to raise the threshold or “cut-off” detection level to the mg/ml range by reducing the sensitivity of the solid support for the analyte, typically no dilution of the sample is required in order to achieve useful results. Thus, in an aspect, the present invention explicitly excludes a step of dilution of the sample before application of the sample to the solid support described herein.

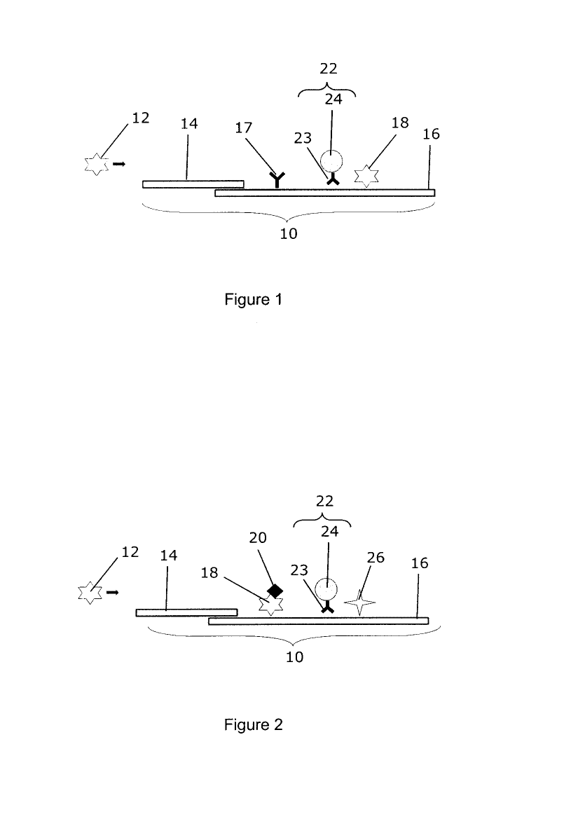

A first aspect of the solid support and method is shown in FIG. 1. The solid support 10 is for detection of an analyte 12. The solid support 10 is, in an aspect, a membrane array comprising a separation membrane 14 and an analytical membrane 16. The analytical membrane 16 comprises an immobilized blocking capture reagent 17 that will bind to and capture an amount of analyte 12 in the sample. Downstream of the blocking capture reagent 17 is a conjugate 22, which comprises an analyte-binding molecule 23 conjugated to a detectable label 24. Downstream of the conjugate 22 is an immobilized competitive analyte 18. The competitive analyte 18 is identical or sufficiently identical to the analyte 12 such that the analyte 12 and the competitive analyte 18 compete for binding to the conjugate 22.

In use, a sample is applied to the solid support 10 at one end. In an aspect, the solid support is a membrane array comprising a separation membrane 14 and an analytical membrane 16 and the sample is applied at the separation membrane 14. The sample will flow laterally along the solid support 10 from the separation membrane 14 into the analytical membrane 16 through capillary action. The sample will first meet the blocking capture reagent 17, which will capture and immobilize a predetermined amount of analyte 12 in the sample, effectively reducing the amount of analyte 12 that will flow further down the solid support. The sample will continue moving along the solid support 10 until it meets the conjugate 22 and the competitive analyte 18. Any remaining analyte 12 in the sample will compete with the competitive analyte 18 for binding to the conjugate 22, forming complexes of analyte 12 and conjugate 22 and/or competitive analyte 18 and conjugate 22, depending upon the amount of analyte 12 in the sample.

If the analyte 12 is absent or present in the sample in an amount that is below the predetermined threshold, the analyte 12 will not have been present in a sufficient amount to out-compete the competitive analyte 18 for binding to the conjugate 22. In this case, the conjugate 22 will bind to the competitive analyte 18 in an amount sufficient to result in a positive signal, indicating that the analyte 12 is absent from the sample or is present in the sample in an amount that is below the predetermined threshold.

If, on the other hand, the analyte 12 is present in the sample in an amount that is equal to or greater than the predetermined threshold, the analyte 12 will have been present in a sufficient amount to out-compete the competitive analyte 18 for binding to the conjugate 22. In this case, the conjugate 22 will not bind to the competitive analyte 18 in an amount sufficient to result in a positive signal, indicating that the analyte 12 is present in the sample in an amount that is equal to or greater than the predetermined threshold.

A second aspect of the solid support and method is shown in FIG. 2. The solid support 10 is for detection of an analyte 12. The solid support 10 is, in an aspect, a membrane array comprising a separation membrane 14 and an analytical membrane 16.

The analytical membrane 16 comprises a competitive analyte 18 coupled to a first member 20 of a binding pair. Downstream of the competitive analyte 18 is a conjugate 22, which comprises an analyte-binding molecule 23 conjugated to a detectable label 24. The competitive analyte 18 is identical or sufficiently identical to the analyte 12 such that the analyte 12 and the competitive analyte 18 compete for binding to the conjugate 22.

Downstream of the conjugate 22, is a second member 26 of the binding pair, also referred to as a capture reagent because it will bind to and capture the first member 20 of the binding pair. It will be understood that the solid support 10 described herein utilizes a combined sandwich/competitive assay format, in which the analyte 12 and competitive analyte 18 compete for binding with the conjugate 22. Additionally, if the analyte 12 is present below the predetermined threshold, as will be described, the second member 26 of the binding pair and the conjugate 22 will form a “sandwich” with the competitive analyte 18 and the first member of the binding pair 20.

The solid support 10 is designed to have a predetermined threshold for detection of the analyte 12, meaning that the solid support 10 will yield a positive result through the detectable label 24 when the analyte 12 is present in the sample at a level that is below the predetermined threshold. While typical methods of adjusting the threshold of a solid support include varying reagent concentrations, membrane pore size, antibody affinity, and so one, it has now been found that the predetermined threshold can be further adjusted based upon at least two additional novel factors.

First, the first member 20 and the second member 26 of the binding pair can be selected based upon their affinity for one another. By selecting a binding pair with a high affinity for one another, the predetermined threshold can be increased. For example, biotin and streptavidin have a particularly strong affinity for one another. Therefore, using biotin as the first member 20 of the binding pair and streptavidin as the second member 26 of the binding pair will yield a solid support 10 with a higher predetermined threshold than if a binding pair with a lower affinity was chosen.

Second, the distance between the competitive analyte 18 and the conjugate 22, as well as the distance between the conjugate 22 and the second member 26 of the binding pair can be varied in order to adjust the predetermined threshold of the solid support 10. As the competitive analyte 18 and the conjugate 22 are placed closer to one another, the distance that the competitive analyte 18 must travel before reaching the conjugate 22 is decreased. This decrease in the distance by which the competitive analyte 18 must travel before reaching the conjugate 22 allows a lesser degree of diffusion of the competitive analyte 18 within the solid support, thereby effectively maintaining the concentration of the competitive analyte 18 that reaches and reacts with the conjugate 22 at a high level. Additionally, as the conjugate 22 and the second member 26 of the binding pair are placed closer to one another, the diffusion of the competitive analyte 18 is further reduced during the reaction process with conjugate 22 due to reduced reaction time. As the concentration of analyte 12 present in the sample is always constant and not affected by diffusion, these variables together lead to a comparative increase in the predetermined threshold of the solid support 10.

It will be understood that this second aspect of the solid support and method as shown in FIG. 2 may also comprise a blocking capture reagent 17 upstream of the competitive analyte 18.

In use, a sample is applied to the solid support 10 at one end. In an aspect, the solid support is a membrane array comprising a separation membrane 14 and an analytical membrane 16 and the sample is applied at the separation membrane 14. The sample will flow laterally along the solid support 10 from the separation membrane 14 into the analytical membrane 16 through capillary action. The sample will first meet the competitive analyte 18 bound to the first member 20 of the binding pair. The competitive analyte 18 will then be mobilized and will move along the solid support 10 with the sample until the sample meets the conjugate 22, comprising the analyte-binding molecule 23 bound to the detectable label 24. Any analyte 12 in the sample will compete with the competitive analyte 18 for binding to the conjugate 22, forming complexes of analyte 12 and conjugate 22 and/or competitive analyte 18 and conjugate 22, depending upon the amount of analyte 12 in the sample. The sample and complexes will continue to flow along the analytical membrane until they meet the second member 26 of the binding pair.

If the analyte 12 is absent or present in the sample in an amount that is below the predetermined threshold, the analyte 12 will not have been present in a sufficient amount to out-compete the competitive analyte 18 for binding to the conjugate 22. Due to the affinity between the first and second members 20, 26 of the binding pair, even a small amount of competitive analyte 18 and conjugate 22 complexes will bind to the second member 26 of the binding pair and result in a positive signal, indicating that the analyte 12 is absent from the sample or is present in the sample in an amount that is below the predetermined threshold.

If, on the other hand, the analyte 12 is present in the sample in an amount that is equal to or greater than the predetermined threshold, the analyte 12 will have been present in a sufficient amount to out-compete the competitive analyte 18 for binding to the conjugate 22, such that few or no competitive analyte 18 and conjugate 22 complexes will bind to the second member 26 of the binding pair. In this way, there will be no signal from the label 24 within the conjugate 22 as it will not have bound to the second member 26 of the binding pair in an amount sufficient to produce a signal, indicating that the analyte 12 is present in the sample in an amount that is equal to or greater than the predetermined threshold.

The sample for use in the solid support 10 may be any bodily fluid. In an aspect, the sample is selected from the group consisting of whole blood, serum, plasma, urine, saliva, sweat, spinal fluid, semen, tissue lysate, milk, colostrum, and combinations thereof. In a specific aspect, the sample is whole blood. Alternatively, the sample may be milk or colostrum. Moreover, the sample may be derived from any source but is typically derived from a mammal. The term “mammal” refers to any animal classified as a mammal, including humans, other higher primates, domestic and farm animals, and zoo, sports, or pet animals, such as dogs, cats, cattle, horses, llamas, alpacas, sheep, pigs, goats, rabbits, etc. Typically, the mammal is a cow, horse, llama, or alpaca. For example, the sample may be whole blood derived from a newborn farm animal, such as a cow or horse, in order to determine whether that animal is in need of immune transfer. The sample may alternatively be derived from the milk or colostrum of the mother of that newborn farm animal to determine the quality of the mother's milk or colostrum with respect to whether or not the mother will be capable of providing the newborn animal with sufficient antibodies. It is contemplated that both the newborn animal and the mother be tested in order to determine the overall likelihood of that animal requiring immune transfer.

The sample volume to be tested can be varied by modifying the size, porosity, and other variables within the solid support. Typically, the sample volume is small, such as the size of about a drop of blood, such as about 10 to about 50 μl. In a specific aspect, the sample volume is about 35 μl.

The time needed for the sample to flow from the point of contact on the solid support to the end of the solid support, yielding a test result, can also be varied by modifying various variables within the solid support. Typically the time is less than about 30 minutes, such as, for example, less than about 25 minutes, less than about 20 minutes, less than about 15 minutes, less than about 10 minutes, less than about 5 minutes, or less than about 2 minutes.

The analyte to be detected is typically an analyte that is ubiquitously found in relatively high amounts in samples to be tested. Generally, the question to be answered is not whether or not the analyte is present in the sample but, rather, whether the analyte is present in the sample above or below a threshold amount. Typically, the threshold amount is high, such as in the mg/ml range, such as at least about 1 mg/ml, 3 mg/ml, 5 mg/ml, 10 mg/ml, 15 mg/ml, 20 mg/ml, 25 mg/ml, 30 mg/ml, 40 mg/ml, or 50 mg/ml. The threshold amount may alternatively be measured in μM quantities and could be at least about 10 μM, 50 μM, 100 μM, 150 μM, 200 μM, 250 μM, or 300 μM. Analytes that are present in such high amounts include immunoglobulins, such as IgA, IgD, IgE, IgG, or IgM, albumin, haemoglobin, or C-reactive protein (CRP). In a specific aspect, the analyte is IgG.

The blocking capture reagent may be any reagent capable of binding to the analyte of interest to thereby reduce the concentration that moves forward along the solid support. The blocking capture reagent may be specific to the analyte of interest and be, for example, an antibody. Alternatively, it may be non-specific to the analyte of interest and may bind to several molecules in the sample. In this case, the blocking capture reagent may be, for example, protein A or G.

The competitive analyte is generally identical to the analyte to be detected. However, it will be understood that any competitive analyte that is capable of competing with the analyte to be detected for binding to the conjugate is contemplated herein. For example, if the analyte is bovine IgG, the competitive analyte could also be bovine IgG. Alternatively, it could be, for example, an Fc fragment of bovine IgG that competes for binding with the conjugate that is specific to the Fc region.

Similarly, the analyte-binding molecule within the conjugate may be any molecule that binds to both the analyte and the competitive analyte. Typically, the analyte-binding molecule is an antibody, such as an anti-IgG antibody. However, the conjugate could also be a peptide or a small molecule, for example. Additionally, if the analyte to be detected is an antibody, the analyte-binding molecule may be, for example, protein A, protein G, protein A/G, or protein L, native or recombinant.

The coupling of the conjugate to the second member of the binding pair will result in a line that is visible if the analyte is absent from the sample or present in the sample below the threshold amount, by virtue of the label on the conjugate. Typically, the label on the conjugate is directly visible to the naked eye upon a positive test result. To this regard, the label may be a metal label, an enzyme label, or a coloured latex bead, for example. Gold is a commonly used metal label and generally has a particle size of from about 20 to about 65 nm. Additionally, it is understood that a gold signal can be enhanced to become readily visible by the use of a reducible silver salt that deposits as a visible product, such as silver lactate, in combination with a reducing agent, such as hydroquinone. The metallic silver forms a readily discernible black deposit around each particle of gold.

Alternatively, the label may not be directly visible to the naked eye upon a positive test result, for example if a fluorescent label is employed. In such a situation, the result of the test will be determined by use of a reader, for example.

Additionally, a second line may form on the solid support as a control to indicate that the test is complete, regardless of whether the result is positive or negative, as is well known. Moreover, the conjugate may function itself as a procedural control. For example, if the conjugate comprises a coloured label, a line will be evident on the solid support before the test is run. After a sample is applied to the solid support, the conjugate will be mobilized and the intensity of the first line will decrease accordingly, indicating that the test is working.

The solid support and method may be qualitative, semi-quantitative, or quantitative. In a qualitative assay, a “yes” or “no” answer is provided, meaning the analyte is present at or above the predetermined threshold or it is not. In a semi-quantitative assay, a second line is produced on the solid support, generally downstream of the signal line containing the capture reagent, wherein the intensity of the second line is constant or inversely proportional to the intensity of the signal line. For example, if the conjugate contains a monoclonal antibody the second line may comprise an antibody against that monoclonal antibody. Any free monoclonal antibody will bind at the second line and emit a signal. If the analyte is absent or present in the sample below the predetermined threshold, a second line will form at the signal line, where the second member of the binding pair is located, as complexes between the conjugate and the competitive analyte will bind there. The higher the level of analyte in the sample, the weaker this second line will be. In comparing the first and second lines, an end user can estimate the level of the analyte in the sample. In a quantitative assay, the intensity of the signal line could be measured by machine to convert the intensity to a corresponding analyte concentration.

The distance between the competitive analyte and the conjugate as well as the distance between the conjugate and the second member of the binding pair may be varied to adjust the predetermined threshold of the solid support, as has been described. It will be understood that these distances will vary depending upon the size of the solid support and the amount of sample it is designed to receive. In an aspect, the conjugate is placed immediately upstream of the second member of the binding pair and/or the competitive analyte is placed immediately upstream of the conjugate. In another aspect, the conjugate and the second member of the binding pair and/or the competitive analyte and the conjugate may be separated by less than about 5 mm, less than about 4 mm, less than about 3 mm, less than about 2 mm, or less than about 1 mm in a solid support designed to receive from about 5 μl to about 50 μl of sample. In a specific example, the competitive analyte, the conjugate, and the second member of the binding pair are each about 2 mm apart in a solid support designed to receive about 35 μl of sample.

The first and second members of the binding pair have been described above as being, for example, biotin and streptavidin. It will be understood that other binding pairs may be selected for use in the present invention, wherein the affinity of the members of the binding pair for one another is used in an aspect to set the predetermined threshold of the solid support. For example, binding pairs that may be used also include biotin and avidin or any known high affinity antigen/antibody binding pair.

The solid support has been described above as being a membrane array comprising a separation membrane and an analytical membrane. However, it will be understood that any solid support format may be used in the present invention. For example, a separation membrane is not required and may be explicitly excluded from the present invention. In a specific aspect, the present invention is for use in any one or more of the solid supports described in U.S. Pat. Nos. 7,785,865, 7,883,899, and 8,119,393 and U.S. Patent Application Publication Nos. 2005/0244985, 2010/0137145, 2010/0323433, 2011/0189791, and 2011/0287461, each of which is incorporated herein by reference in its entirety. A membrane is not required for use of the solid support described herein. To this regard, the solid support may be, for example, a capillary channel such as that commercialized by Biosite (see, for example U.S. Pat. No. 6,669,907, incorporated herein by reference in its entirety) or it may be a microfluidic channel.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. It will be understood that in describing any feature of the present invention with the term “comprising” or its equivalents, the present invention also includes variants wherein it “consists of” the recited features. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The above disclosure generally describes the present invention. A more complete understanding can be obtained by reference to the following specific Examples. These

Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1—Comparative Example

A solid support for carrying out a competition assay for detection of the analyte C-reactive protein (CRP) was prepared, comprising a separation membrane and an analytical membrane, with the analytical membrane being downstream of the separation membrane. A colloidal gold conjugate solution with a final OD 3 at 540 nm was prepared from 40 nm gold particles (British Biocell International) conjugated to an anti-CRP monoclonal antibody (Hytest). The separation membrane (Whatman) was coated with the colloidal gold conjugate solution and was then freeze-dried to remove water. The analytical membrane was made from nitrocellulose (Millipore) having a pore size of 8 μm. The analytical membrane was impregnated with capture solution containing 1.5 mg/ml CRP followed by incubation at 37° C. for 30 minutes to dry the analytical membrane. The separation membrane and the analytical membrane were covered by a transparent polyester tape (Adhesive Research) and were supported by polystyrene backing tape (G&L Precision Die Cutting, Inc.).

To conduct the test, 35 μl of human serum was applied to the separation membrane. After approximately 15 minutes, the test was complete. Using this conventional design, the threshold for CRP detection was at most about 10 μg/ml.

### Example 2

A solid support for carrying out a modified competition assay for detection of the analyte IgG was prepared, comprising a separation membrane (Whatman) and an analytical membrane made from nitrocellulose (Millipore) having a pore size of 5 μm. A colloidal gold conjugate solution with a final OD 16 at 540 nm was prepared from 60 nm gold particles (British Biocell International) conjugated to a goat anti-bovine IgG polyclonal antibody

(BiosPacific) and included gelatin as a blocking agent. A blocking capture solution was prepared and contained 2.2 mg/ml of the same goat anti-bovine IgG polyclonal antibody used in the conjugate solution. Finally, a capture solution containing 3.5 mg/ml bovine IgG was prepared. The three solutions were impregnated into the analytical membrane, with the conjugate solution being downstream of the blocking capture solution and the capture solution being downstream of the conjugate solution. In particular, the capture solution was located less than about 2 mm downstream of the conjugate solution. The analytical membrane was then incubated at 37° C. for 40 minutes to dry the reagents. The analytical membrane was covered by a transparent polyester tape (Adhesive Research) and was supported by polystyrene backing tape (G&L Precision Die Cutting, Inc.).

To conduct the test, 35 μl of bovine serum was applied to the separation membrane. After approximately 15 minutes, the test was complete. Using this design, the threshold for IgG detection was about 3 mg/ml.

### Example 3

A solid support for carrying out a combined sandwich/competition assay for detection of the analyte IgG was prepared, comprising a separation membrane (Whatman) and an analytical membrane made from nitrocellulose (Millipore) having a pore size of 5 μm. A colloidal gold conjugate solution with a final OD 16 at 540 nm was prepared from 60 nm gold particles (British Biocell International, BBI) conjugated to a goat anti-bovine IgG polyclonal antibody (BiosPacific) and included gelatin as a blocking agent. Biotinylated bovine IgG corresponding to the IgG to be detected was prepared at a concentration of 3 mg/ml. Finally, a capture solution containing 3 mg/ml streptavidin (IPOC Inc.) was prepared. The three solutions were impregnated into the analytical membrane, with the conjugate solution being downstream of the biotinylated bovine IgG and the capture solution being downstream of the conjugate solution. In particular, the capture solution was located less than about 2 mm downstream of the conjugate solution. The analytical membrane was then incubated at 37° C. for 40 minutes to dry the reagents. The analytical membrane was covered by a transparent polyester tape (Adhesive Research) and was supported by polystyrene backing tape (G&L Precision Die Cutting, Inc.).

To conduct the test, 35 μl of bovine serum was applied to the separation membrane. After approximately 15 minutes, the test was complete. Using this design, the threshold for IgG detection was at least about 30 mg/ml.

The above disclosure generally describes the present invention. Although specific terms have been employed herein, such terms are intended in a descriptive sense and not for purposes of limitation.

All publications, patents and patent applications are herein incorporated by reference in their entirety to the same extent as if each individual publication, patent or patent application was specifically and individually indicated to be incorporated by reference in its entirety.

Although exemplary embodiments of the invention have been described herein in detail, it will be understood by those skilled in the art that variations may be made thereto without departing from the spirit of the invention or the scope of the appended claims.

1. A solid support for detecting the presence of an analyte in a sample at or above a predetermined threshold, the solid support comprising:
a competitive analyte within said solid support, wherein said competitive analyte is coupled to a first member of a binding pair;
a labelled conjugate within said solid support, downstream of said competitive analyte, wherein said analyte and said competitive analyte compete for binding to said conjugate; and
a capture reagent immobilized within said solid support, downstream of said conjugate, wherein said capture reagent comprises a second member of the binding pair;
wherein the affinity of the first and second members of the binding pair for one another and the distance between the conjugate and the capture reagent and/or the conjugate and the competitive analyte within the solid support are selected so as to increase the threshold of the solid support for the analyte.

2. The solid support of claim 1, wherein the analyte is an immunoglobulin.

3. The solid support of claim 2, wherein the immunoglobulin is IgG.

4. The solid support of claim 1, wherein the conjugate is an antibody specific for the analyte.

5. The solid support of claim 1, wherein the first member of said binding pair is biotin and the second member of said binding pair is streptavidin or avidin.

6. The solid support of claim 1, wherein the sample is a biological sample selected from the group consisting of whole blood, serum, plasma, urine, milk, and colostrum.

7. The solid support of claim 1, wherein the predetermined threshold is at least about 1 mg/ml, 3 mg/ml, 5 mg/ml, 10 mg/ml, 15 mg/ml, 20 mg/ml, 25 mg/ml, 30 mg/ml, 40 mg/ml, or 50 mg/ml.

8. The solid support of claim 1, further comprising a blocking capture reagent upstream of said competitive analyte.

9. A solid support for detecting the presence of an analyte in a sample at or above a predetermined threshold, the solid support comprising:
a blocking capture reagent immobilized within said solid support, wherein said blocking capture reagent binds to the analyte in the sample and thereby reduces the concentration of the analyte that is mobile;
a labelled conjugate within said solid support, downstream of said blocking capture reagent; and
a capture reagent immobilized within said solid support, downstream of said conjugate, wherein said capture reagent is a competitive analyte and wherein said analyte and said competitive analyte compete for binding to said conjugate;
wherein the distance between the conjugate and the capture reagent within the solid support is selected so as to increase the threshold of the solid support for the analyte.

10. A one-step method for detecting an analyte in a sample, the method comprising applying the sample to the solid support of claim 1, wherein a positive signal indicates that the analyte is absent or present in an amount below the predetermined threshold.

11. A one-step method for determining whether a newborn mammal is in need of immune transfer, the method comprising applying a biological sample from the newborn mammal or its mother to a solid support, said solid support comprising:
mammalian IgG coupled to a first member of a binding pair;
labelled anti-mammalian IgG antibody downstream of said mammalian IgG; and
a second member of the binding pair downstream of said anti-mammalian IgG;
wherein the affinity of the first and second members of the binding pair for one another and the distance between the anti-mammalian IgG antibody and the second member of the binding pair and/or the anti-mammalian IgG and the mammalian IgG are selected such that a positive signal results when the mammal has an IgG level at or below a predetermined threshold in the mg/ml range, indicating that the mammal is in need of immune transfer.

12. The method of claim 11, wherein the first member of said binding pair is biotin and the second member of said binding pair is streptavidin or avidin.

13. The method of claim 11, wherein the sample is derived from the newborn mammal and is selected from the group consisting of whole blood, serum, plasma, and urine.

14. The method of claim 11, wherein the sample is derived from the mother of the newborn mammal and is selected from the group consisting of milk and colostrum.

15. The method of claim 1, wherein the mammal is a dog, cat, cow, horse, llama, alpaca, sheep, pig, goat, or rabbit.

16. The method of claim 1, wherein the predetermined threshold is about 10 mg/ml.
